(19) United States
(12) Patent Application Publication
(10) Pub. No.: US 2004/0241120 A1
(43) Pub. Date: Dec. 2, 2004

(54) Cosmetic composition comprising silicate particles in an oil-in-water emulsion carrier

(76) Inventors: Francoise Pataut, Paris (FR); Cecile Nocerino, Paris (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/479,345

(22) PCT Filed: May 31, 2002
(86) PCT No.: PCT/FR02/01846

(30) Foreign Application Priority Data
May 31, 2001 (FR) 0107158

(51) Int. Cl. A61K 7/06

(52) U.S. Cl. 424/70.1

(57) ABSTRACT

The invention concerns a cosmetic composition, in particular for the hair, comprising, in a cosmetically acceptable medium of the oil-in-water emulsion type, solid particles containing at least 10 wt. % of at least a silicate. The invention is characterised in that: the number average primary size of globules forming the oil phase ranges between 1 and 40 μm; the silicate particles contain less than 1 wt. % of aluminium; the composition contains between 5 and 50 wt. % relative to the total weight of the composition of at least a C1-C4 alcohol.

The present invention relates to a cosmetic composition, especially for the hair, containing silicate particles in a vehicle of the oil-in-water emulsion type. It further relates to a method of cosmetic treatment for the hair comprising the application of this composition, and to its use as a hair product.

Numerous cosmetic compositions for the hair are known to contain a film-forming polymer which, when applied in the form of a solution, gives rise, after evaporation of the solvent, to the formation of a film that coats the hair. It is known that many of these film-forming polymers used in cosmetic compositions for the hair share in particular the property of strengthening the hold of the hair. These film-forming polymers can be deposited via lotions that may or may not be propelled by means of an aerosol device, or via foams, gels or emulsions.

However, the hold obtained with these film-forming polymers does not generally last very long and the feel imparted is not always very natural, so there is a need for compositions which give keratin fibers a natural and durable hold while at the same time preserving or enhancing the natural gloss of the fibers.

It has already been proposed to use particles in rinsed compositions in order to improve the feel and appearance of the hair. By way of illustration, patent U.S. Pat. No. 5,334,376 proposes to add calcium carbonate particles to hair conditioning compositions that contain a silicone, a fatty alcohol and an amide.

Patent application DE 199 46 784 further proposes to use particles of various oxides, hydroxides, carbonates, silicates or phosphates in hair compositions in order to reduce the greasy appearance of the hair. In the majority of cases, provision is made to combine these particles with the conventional ingredients of shampoos.

Patent EP 666 071, in the name of the Applicant, proposes cosmetic compositions for photoprotection of the skin or hair which contain, in a cosmetically acceptable vehicle of the ultrafine oil-in-water emulsion type in which the size of the globules constituting the oily phase is between 100 nm and 1 μm, at least one alkali metal and/or alkaline earth metal silicate in the presence of photoprotective metal oxide nanoparticles. Nevertheless, these compositions have the disadvantage of sometimes make the hair heavy, loaded and difficult to disentangle, and dull.

The Applicant has discovered, surprisingly and unexpectedly, that by choosing an emulsion of the oil-in-water type in which the size of the oil globules forming the oily phase is within a defined range, and in which the aqueous phase contains a given amount of alcohol in combination with silicate particles, it is possible to improve the results obtained in the prior art with cosmetic products, especially for the hair, in terms of cosmetic properties and hair shaping properties.

The invention relates to a cosmetic composition, especially for the hair, comprising, in a cosmetically acceptable medium of the oil-in-water emulsion type, solid particles containing at least 10% by weight of at least one silicate, characterized in that:

(a) the number-average primary size of the globules constituting the oily phase is between 1 and 40 μm;

(b) the silicate particles contain less than 1% by weight of aluminum; and

(c) the composition contains between 5 and 50% by weight, based on the total weight of the composition, of at least one C₁ to C₄ alcohol.

The compositions according to the invention also have a reduced greasiness and give keratin fibers an excellent level of gloss.

The present invention further relates to a method of cosmetic treatment for the hair using the composition according to the invention.

The invention further relates to the use of a composition such as defined above as a hair product, especially for fixing and/or shaping the hair.

Further objects, characteristics, features and advantages of the invention will become even more clearly apparent from the description and the various examples which follow.

### SILICATE PARTICLES

Advantageously, the solid particles have a number-average primary size of between 2 nm and 1 μm.

Preferably, the particles contain between 0 and 1% by weight of aluminum.

The particles containing at least 10% by weight of at least one silicate have a number-average primary size preferably of between 5 and 500 nm and particularly preferably of between 10 and 250 nm.

The particles according to the invention can have any shape, for example that of spheres, flakes, needles or platelets, or totally random shapes.

Preferred silicates are those of sodium, magnesium and/or lithium, such as the compounds marketed by Laporte under the names LAPONITE XLG and LAPONITE XLS.

In terms of the present invention, “primary particle size” is understood as meaning the maximum dimension that it is possible to measure between two diametrically opposite points of an individual particle. The size can be determined e.g. by transmission electron microscopy, or from measurement of the specific surface area by the BET method, or using a laser granulometer.

In the case where the particles are formed of silicate and other fillers, the silicate is present in the free state and does not form chemical bonds with the other fillers. Thus there is an alloy between the silicate and other fillers, especially with metal or metalloid oxides, obtained in particular by thermal fusion of these different constituents.

If the particles containing at least 10% by weight of at least one silicate also comprise a metal or metalloid oxide, the latter is selected especially from silicon, boron and aluminum oxides.

The particles contain preferably at least 50% by weight of silicate and particularly preferably at least 70% by weight, those containing more than 90% by weight of silicate being particularly preferred according to the present invention.

A suitable silicate for the compositions of the present invention can be of natural or synthetic origin.

The particles containing silicate according to the invention are used especially in an amount of between 0.05% and 10% by weight and preferably of between 0.5% and 2.5% by weight, based on the total weight of the composition.

The composition according to the invention can also contain other types of particles, for example titanium oxide, zinc oxide or aluminum oxide particles.

Depending on the extent of their lipophilic or, conversely, hydrophilic character, the silicates may be present either in the oily phase of the emulsion, or in the aqueous phase, or in both phases simultaneously.

### EMULSION

The oily phase forming part of the composition of the emulsions according to the invention is not of a critical nature and can thus consist of any of the compounds already known in general terms as suitable for the manufacture of emulsions of the oil-in-water type. In particular, these compounds can be selected, individually or in mixtures, from various fatty substances, oils of vegetable, animal or mineral origin, natural or synthetic waxes, and the like.

The following may be mentioned in particular among the oils capable of forming part of the composition of the oily phase:

mineral oils, such as paraffin oil and liquid paraffin,

oils of animal origin, such as perhydrosqualene,

oils of vegetable origin, such as sweet-almond oil, avocado oil, castor oil, olive oil, jojoba oil, sesame oil, groundnut oil, grape seed oil, colza oil, copra oil, hazelnut oil, shea butter, palm oil, apricot kernel oil, calophyllum oil, rice bran oil, maize germ oil, wheat germ oil, soybean oil, sunflower oil, evening primrose oil, safflower oil, passiflora oil and rye oil, and

synthetic oils, such as purcellin oil, butyl myristate, isopropyl myristate, cetyl myristate, isopropyl palmitate, isopropyl adipate, ethylhexyl adipate, butyl stearate, hexadecyl stearate, isopropyl stearate, octyl stearate, isocetyl stearate, decyl oleate, hexyl laurate, propylene glycol dicaprylate, esters derived from lanolic acid, such as isopropyl lanolate and isocetyl lanolate, isoparaffins and poly-α-olefins.

The following may also be mentioned as other oils capable of being used in the emulsions according to the invention: C₁₂-C₁₅ fatty alcohol benzoates (Finsolv TN from FINETEX), fatty alcohols, such as lauryl, cetyl, myristyl, stearyl, palmityl and oleyl alcohols and 2-octyldodecanol, acetylglycerides, octanoates and decanoates of alcohols and polyalcohols, such as those of glycol and glycerol, ricinoleates of alcohols and polyalcohols, such as those of cetyl alcohol, fatty acid triglycerides, such as caprylic/capric triglycerides and triglycerides of C₁₀-C₁₈ saturated fatty acids, fluorinated and perfluorinated oils, lanolin, hydrogenated lanolin, acetylated lanolin and, finally, volatile or non-volatile silicone oils.

Of course, the oily phase can also contain one or more conventional lipophilic cosmetic adjuvants, especially those which are already conventionally used in the manufacture and formulation of cosmetic compositions.

Advantageously, the mean size of the liquid particles (or globules) of oily phase in the aqueous continuous phase must be between 1 and 20 μm. Particularly preferably, the size distribution of the oily globules is such that the majority of said globules (i.e. at least 90% by number) have a size between the limits indicated above.

The size of the oily globules can easily be determined by optical microscopy.

The stable compositions according to the invention can be prepared by any suitable process known per se which essentially involves the introduction of mixed silicates into an oil-in-water emulsion. They can thus be obtained either by introducing the silicate(s) into a preprepared emulsion (first variant), which itself may have been obtained by any means known per se (ultrasound, mixers/homogenizers, phase inversion, or the like), or by integrating this silicate introduction step into at least one of the steps of a conventional emulsion preparation process (second variant); in the latter variant, the only difference between the emulsions obtained and those which would be obtained by the same process but without using silicates is the presence of said silicates in said emulsions. Consequently, to obtain emulsions according to the invention with desired characteristics of chemical composition (other than silicates) and structure, it suffices to carry out a known and conventional process for the preparation of O/W emulsions which is known to produce emulsions with the desired characteristics, but into which a silicate introduction step will additionally have been integrated. Of course, this rule of correspondence between emulsions not containing silicates and those containing silicates also applies by analogy to the first variant referred to above.

In particular, it is possible to produce emulsions without surfactants (so-called Pickering emulsions).

The aqueous continuous phase can consist e.g. of water or a mixture of water and C₁ to C₄ alcohol(s) such as ethanol, isopropanol or butanol (aqueous-alcoholic solution) and it can of course also contain conventional water-soluble cosmetic adjuvants.

The aqueous continuous phase consists of a mixture of ethanol and water with an ethanol content of between 5 and 50% and preferably of between 10 and 30%, based on the total weight of the composition. It can optionally contain one or more other C₂ to C₈ alcohols or polyols.

### COMPOSITION

By way of indication, the formulations according to the invention generally have the following compositions:

aqueous phase: 20 to 90% and preferably 30 to 80% by weight, based on the total weight of the composition;

oily phase: 2 to 40% and preferably 5 to 30% by weight, based on the total weight of the composition;

silicates: 0.01 to 10% and preferably 0.5 to 3% by weight, based on the total weight of the composition.

The compositions according to the invention can contain, in addition to the combination defined above, viscosity regulators such as electrolytes or thickeners (associative or non-associative). Compounds which may be mentioned in particular are sodium chloride, sodium xylenesulfonate, scleroglucans, xanthan gums, fatty acid alkanolamides, alkyl ether carboxylic acid alkanolamides optionally ethoxylated with up to 5 mol of ethylene oxide, such as the product marketed under the name “AMINOL A15” by CHEM Y, crosslinked polyacrylic acids and crosslinked acrylic acid/C₁₀-C₃₀-alkyl acrylate copolymers. These viscosity regulators are used in the compositions according to the invention in proportions ranging up to 10% by weight, based on the total weight of the composition.

The compositions according to the invention can also contain up to 5% of pearlescent agents or opacifiers well known in the state of the art, for example fatty alcohols above C₁₆, acylated derivatives with a fatty chain, such as ethylene glycol or polyethylene glycol monostearates or distearates, and ethers with fatty chains, for example distearyl ether or 1-(hexadecyloxy)-2-octadecanol.

The compositions according to the invention can optionally also contain at least one additive selected from siliconized or non-siliconized sun filters, surfactants, anionic, non-ionic or amphoteric polymers, proteins, protein hydrolyzates, hydroxy acids, vitamins, provitamins, such as panthenol, organomodified or non-organomodified, crosslinked or non-crosslinked, volatile or non-volatile linear or cyclic silicones, and any other additive conventionally used in the cosmetics sector which does not affect the properties of the compositions according to the invention.

It may be of value to introduce one or more anionic, amphoteric, cationic or non-ionic fixing polymers into the compositions in order to strengthen the holding effect.

If the compositions according to the invention contain at least one surfactant, these are present preferably at a concentration of between 0.05 and 4% by weight, based on the total weight of the composition, and particularly preferably at a concentration of between 0.05 and 2% by weight, based on the total weight of the composition.

Of course, those skilled in the art will take care to choose this (these) optional complementary compounds and/or their amounts in such a way that the advantageous properties intrinsically attached to the combination according to the invention remain unaffected or substantially unaffected by the addition(s) envisaged.

These additives are optionally present in the composition according to the invention in proportions ranging from 0.00001 to 20% by weight, based on the total weight of the composition. The amount of each additive is easily determined by those skilled in the art in accordance with its nature and its function.

These compositions can take the form of more or less thickened liquids or of creams or gels and are suitable mainly for hair styling and for the care of keratin fibers, particularly the hair.

These compositions can be used in a rinsed or, preferably, non-rinsed mode.

Concrete examples will now be given to illustrate the invention, but without implying a limitation.

### EXAMPLES

The following three compositions are prepared:

| Composition 1 | |
|---|---|
| Laponite XLS (1) | 2% |
| Water | 58% |
| Ethanol | 20% |
| Isopropyl myristate | 20% |
| Composition 2 | |
| Laponite XLS (1) | 2% |
| Water | 58% |
| Ethanol | 20% |
| Jojoba oil | 20% |
| Composition 3 | |
| Laponite XLS (1) | 2% |
| Water | 58% |
| Ethanol | 20% |
| Copra oil | 15% |
| Phenylated silicone | 5% |

(1) number-average primary size: 25 nm, marketed by Laporte

The composition has a pleasant texture when applied to dry hair. After application, the hair is structured and glossy.

1. A cosmetic composition, comprising, in a cosmetically acceptable oil-in-water emulsion, solid particles comprising at least 10% by weight of at least one silicate, wherein:
the oily phase of the emulsion comprises oil globules having a number-average primary size of between 1 and 40 μm;
the silicate-comprising solid particles comprise less than 1% by weight of aluminum; and
the composition comprises between 5 and 50% by weight, based on the total weight of the composition, of at least one C₁ to C₄ alcohol.

2. The composition as claimed in claim 1, wherein the solid particles comprising at least 10% by weight of silicate have a number-average primary size of between 2 nm and 1 μm.

3. The composition as claimed in claim 1, wherein the solid particles comprise at least 50% by weight of silicate.

4. The composition as claimed in claim 1, wherein the solid particles are 100% silicate.

5. The composition as claimed in claim 1, wherein the solid particles comprising silicate are present in an amount of between 0.01% and 10% by weight, based on the total weight of the composition.

6. The composition as claimed in claim 1, wherein the mean size of the oil globules in the aqueous continuous phase is between 1 and 20 μm.

7. The composition as claimed in claim 1, wherein the amount of C₁ to C₄ alcohol in the composition is between 10 and 30% by weight, based on the total weight of the composition.

8. The composition as claimed in claim 1, wherein the oil is at least one selected from the group consisting of:
mineral oil,
an oil of animal origin,
an oil of vegetable origin,
a synthetic oil,
a C₁₂-C₁₅ fatty alcohol benzoate,
a fatty alcohol,
a acetylglyceride,
a octanoate of an alcohol, a octanate of a polyalcohol, a decanoate an of alcohol, a decanoate of a polyalcohol,
a ricinoleate of an alcohol, a ricinoleate of a polyalcohol,
a fatty acid triglyceride,
a triglyceride of a C₁₀-C₁₈ saturated fatty acid,
a fluorinated oil, a perfluorinated oil,
lanolin, hydrogenated lanolin, acetylated lanolin,
a volatile silicone oil and a non-volatile silicone oil.

9. The composition as claimed in claim 1, wherein the oily phase is present in the composition at a concentration of 2 to 40%, based on the total weight of the composition.

10. The composition as claimed in claim 1, further comprising one or more anionic, amphoteric, cationic or non-ionic fixing polymers.

11. The composition as claimed in claim 1, wherein the silicate is at least one of a silicate of sodium, magnesium or lithium.

12. The composition as claimed in claim 1, further comprising at least one additive selected from the group consisting of siliconized or non-siliconized sun filters; cationic surfactants; anionic, non-ionic, cationic or amphoteric polymers; proteins, protein hydrolyzates, hydroxy acids, vitamins, provitamins, panthenol; and organomodified or non-organomodified, crosslinked or non-crosslinked, volatile or non-volatile linear or cyclic silicones.

13-14. (Canceled).

15. The composition as claimed in claim 1, wherein the solid particles comprising at least 10% by weight of silicate have a number-average primary particle size of between 5 and 500 nm.

16. The composition as claimed in claim 1, wherein the solid particles comprising at least 10% by weight of silicate have a number-average primary particle size of between 10 and 250 nm.

17. The composition as claimed in claim 1, wherein the solid particles comprise at least 70% by weight of silicate.

18. The composition as claimed in claim 1, wherein the solid particles comprise at least 90% by weight of silicate.

19. The composition as claimed in claim 1, wherein the solid particles comprising silicate are present in an amount of between 0.5% and 3% by weight based on the total weight of the composition.

20. The composition as claimed in claim 1, wherein the oily phase is present in the composition at a concentration of 5 to 30% by weight based on the total weight of the composition.

21. A hair care composition comprising the cosmetic composition of claim 1 and one or more carriers or diluents.

22. A method comprising
applying the composition as claimed in claim 1 to hair.

23. A method comprising
applying a composition comprising the cosmetic composition of claim 1 to the hair in an amount effective for at least one of fixing or shaping the hair.
